(19) United States
(12) Patent Application Publication
(10) Pub. No.: US 2013/0123685 A1
(43) Pub. Date: May 16, 2013

(54) ELECTRONICALLY MONITORED INJECTION DEVICE

(71) Applicants: Soren Kragh Jespersen, Fredensborg (DK); Claus Valentin Buschardt, Bagsvaerd (DK); Kim Ejholm Hansen, Allerod (DK); Bo Kvolsbjerg, Helsingor (DK); Bennie Peder Smiszek Pedersen, Haslev (DK); Claus Urup Gjødesen, Kobehavn O (DK); Preben Mikael Nielsen, Holbaek (DK)

(72) Inventors: Soren Kragh Jespersen, Fredensborg (DK); Claus Valentin Buschardt, Bagsvaerd (DK); Kim Ejholm Hansen, Allerod (DK); Bo Kvolsbjerg, Helsingor (DK); Bennie Peder Smiszek Pedersen, Haslev (DK); Claus Urup Gjødesen, Kobehavn O (DK); Preben Mikael Nielsen, Holbaek (DK)

(73) Assignee: Novo Nordisk A/S, Bagsvaerd (DK)

(21) Appl. No.: 13/375,033

(22) PCT Filed: Jun. 3, 2010
(86) PCT No.: PCT/EP2010/057777

§ 371 Date: Feb. 16, 2012

Related U.S. Application Data
(60) Provisional application No. 61/183,748, filed on Jun. 3, 2009.

(51) Int. Cl. A61M 5/315; A61M 5/20

(52) U.S. Cl. CPC A61M 5/31546; A61M 5/20
USPC 604/65

(57) ABSTRACT

Medical injection advice adapted for operating in a dose setting mode and in a dose administration mode, comprising a dosing actuator (124) reversibly actuatable from a first state for operating the device in the dose setting mode to a second state for operating the device in the dose administration mode, a switch (130, 132) adapted to sense positional information associated with the dosing actuator, and a controller coupled to the switch arrangement, wherein electronic mode representation is configured to enter dose setting mode after a time delay following detecting the dosing actuator has moved away from said second state or detecting the dosing actuator has moved into said first state.

The present invention relates to medical injection devices for injecting a portioned doses of a medicament. More specifically, the invention relates to medical injection devices having means for precisely monitoring injection device operation modes.

The invention enables a more efficient use of injection devices while offering improvements with respect to dose delivery accuracy.

### BACKGROUND OF THE INVENTION

In the disclosure of the present invention reference is mostly made to the treatment of diabetes by injection or infusion of insulin, however, this is only a preferred use of the present invention.

In order to permit a patient to administer a proper dose, various mechanical injection devices have been proposed which offer additional electric circuitry for measuring and displaying the size of a dose which will be administered during an injection procedure. Examples of such devices are shown in WO02/064196 and in WO02/092153.

Some mechanical injection devices are equipped with an energy accumulating member such as a spring for the purpose of accumulating mechanical energy during a pre-injection procedure, e.g. by straining a spring during a dose setting procedure, to enable the accumulated energy to drive a drive member during the subsequent injection procedure so as to expel a fixed or an individually set dose from a plunger equipped medicament container. WO2008/037801 disclose such devices wherein additional electric circuitry is provided for detecting the amount of a set dose or the amount of an injected dose.

In injection devices of the type described above, the degree of accuracy of detection of dose sizes may in certain situations not be fully acceptable. Ideally, for injection devices incorporating high precision dose setting and delivery mechanisms, the electronic dose determination should provide the same level of accuracy. However, in prior art devices, the dose determination systems does not provide fully acceptable dose size determination, in particular during certain operating sequences. In particular, in injection devices which are designed to work in a dose setting mode and in a dose administration mode, the synchronism between the various mechanical movements and the detected electronic representation of these movements is not always guaranteed, leading to potential issues having regard to the dose size determination.

Some prior art devices include provisions for assisting the user of the device to select a particular dose size each time the injection device is used, wherein the particular dose size is selected from the complete range of adjustable dose sizes. In manual injection devices, such provision may be implemented by including an adjustable mechanism for indicating when the dose size corresponding to the preset dose is adjusted. Examples are disclosed in U.S. Pat. No. 7,377,913 and WO 2007/107431. For motorized injection devices, WO 2005/097237 discloses an injection device having multiple preset doses to select between by using a set of keys. Also, in U.S. Pat. No. 7,201,741, an injection device is shown, wherein upper and lower limits for appropriate dose sizes may be indicated to the user.

### DISCLOSURE OF THE INVENTION

Having regard to the problems and deficiencies identified above, it is an object of a first aspect of this invention to provide an injection device having improved accuracy in detecting set doses and/or expelled doses.

Also, it is an object of a second aspect of this invention to improve the dose detection accuracy in injection devices being optimized for minimized power consumption and to reduce the requirements for narrow tolerances on the mechanical parts of an injection device.

Also, it is an object of a third aspect of this invention to improve a manual injection device being used by patients following a fixed dose treatment regime and to improve the user guidance provided by the injection device.

In accordance with the FIRST aspect of the invention, a medical injection device is provided for injection of set doses of a medicament from a medicament container. The injection device is adapted for operating in a dose setting mode and in a dose administration mode. The injection device comprises:
a drive member which moves during dose setting mode and/or during dose administration mode, the drive member being adapted to cause the expelling of a dose of medicament from the medicament container during dose administration,
a dose sensor arrangement adapted to sense positional information associated with the drive member,
a dosing actuator movable between a first state and a second state, wherein the dosing actuator, when it is in the first state for operating the device in the dose setting mode to a second state for operating the device in the dose administration mode,
a switch arrangement adapted to sense positional information associated with the dosing actuator to determine whether the injection device is in dose setting mode or dose administration mode, and
a controller coupled to the dose sensor arrangement and to the switch arrangement, the controller being adapted to provide an electronic mode representation of the mode of operation and being adapted to determine the size of a set dose and/or an expelled dose, wherein the size of the set dose and/or the expelled dose is being determined from said positional information in conjunction with the detected electronic mode representation.

In the device, responsive to the dosing actuator moving from the second state to the first state, the electronic mode representation is configured to enter dose setting mode after a time delay following detecting the dosing actuator has moved away from said second state or detecting the dosing acutator has moved into said first state.

Hence, in order to discriminate properly between movements carried out for the dose setting process and movements carried out for the dose administration process said time delay is incorporated into the arrangement for determining the size of a set dose and/or an expelled dose, the accuracy of the determined dose size may be increased by taking into account dynamic effects which occur when the mode of the injection apparatus is switched from one mode to the other

In one embodiment, the injection device includes a dose sensor arrangement which comprises a single sensor arrangement adapted to detect said positional information associated with the drive member during both dose administration as well as during dose setting operations.

In a further embodiment, the controller is adapted to determine both the size of a set dose and the size of an expelled dose.

In particular embodiments, the medical injection device is a manual injection device.

In some embodiments, the device further comprises a spring means configured for storing energy during a pre-injection procedure, and wherein the drive member, during dose administration, is forced by the spring means to expel medication from the container when energy previously stored in said spring means is released.

The spring means may be provided by a spring, such as a compression spring or a torsion spring. Alternatively, the spring means may incorporate a resilient foam material or a compressible gas medium.

In some embodiments, the dosing actuator cooperates with the spring means to release energy when the dosing actuator is moved into its second state, said release of energy being interrupted when the dosing actuator is returned towards its first state. This enables interruption of dose administration during administration of a set dose, i.e. before the complete dose as originally set has been expelled. In other words, the administration of a set dose of a medicament may be paused anytime between start of dose and end of dose providing the possiblity of finalizing the remaining part of the originally set dose by moving the dosing actuator into its second state again.

In particular embodiments, the time delay may be larger than 5 msecs, preferably larger than 10 msecs, preferably larger than 50 msecs, preferably larger than 100 msecs, preferably larger than 250 msecs and more preferably larger than 500 msecs.

In particular embodiments, the time delay is less than 1 sec., preferably less than 750 ms, preferably less than 500 ms, preferably less than 400 ms and more preferably less than 250 ms.

Further, in particular embodiments, the time delay may be determined by an electronic counter which runs from the detection that the dosing actuator has moved away from said second state or has moved into said first state, and wherein the electronic mode representation enters dose setting mode after a particular count has been reached.

The injection device may incorporate a dosage selector which may be rotated relative to a housing of the device in order to set a dose. In device embodiments where, by incorporating an click-mechanism for the dosage selector, the dosage selector is adapted to move in incremental steps corresponding to complete units of a doseable medicament, such as full units of insulin (measured in International Units) or alternatively in fractional units such as half units. In such systems, the resolution of the sensor arrangement may be higher than the incremental steps of the dose setting member so as to provide for a particular accurate dose size determination.

In embodiments where the injection device includes a dose setting mechanism which enables the user to select a particular size of dose to be administered, the spring means may be adapted to be cocked or biased during the dose setting operation. In alternative embodiments, the spring means is adapted to be cocked or biased during an initial operation, such as by manipulating a cap on or off or moving a dosing actuator into an armed position ready for actuation.

The drive member may take form of or include a piston rod for driving forward the piston of the medicament container. The said sensor may be coupled to the drive member to sense movements of the drive member during injection. Alternatively, the said sensor may sense movements of other components of the device which is coupled or associates with the drive member, i.e. which moves in synchronism with the drive member at least during injection. In accordance with the particular design of the drive mechanism, the sensor may sense a linear movement or alternatively a rotary movement or still alternatively a helical movement.

The sensor is coupled to the controller to monitor movements of a component at least during injection, so as to provide positional information relating to the particular component in question. Within the context of this invention, the term “positional information” includes parameters related to the positional data of a component over time.

In certain embodiments, the injection device is configured for storing a log or diary comprising data relating to a plurality of administered dose injections, e.g. expelled dose sizes and time and date of the administered dose etc. Such information may be subsequently recalled from the storage of the device and shown on a display on the device. Further, this information may be communicated to an auxiliary device. As an alternative to storing time and date of particular dose administrations, the device may rely exclusively on a continuous counter, i.e. a counter which do not represent the actual time of the day, whereby relative time stamps between individual dose administrations can be determined and stored. After the stored relative time stamps and the dose sizes associated which each relative time stamp has been transferred to an auxilliary device, the actual date and time for the individual dose administrations can be calculated in the auxilliary device.

In accordance with the SECOND aspect of the invention, a medical injection device is provided for injection of set doses of a medicament from a medicament container, the injection device being adapted for operating in a dose setting mode and in a dose administration mode, the injection device comprising:
a drive member being adapted to expel a dose of medicament from the medicament container during dose administration,
a dose sensor arrangement adapted to detect positional information of the drive member,
a dosing actuator reversibly movable from a first state for operating the device in the dose setting mode to a second state for operating the device in the dose administration mode,
a switch arrangement monitoring the position of the dosing actuator to determine whether the injection device is in dose setting mode or dose administration mode, and
a controller coupled to the dose sensor arrangement and to the switch arrangement to determine the size of a set dose, the dose size being determined from said positional information when the switch arrangement determines that the device is in dose setting mode,
   wherein the switch arrangement is adapted to provide an electronic representation of transition into dose setting mode at a first position and to provide an electronic representation of transition into dose administration mode at a second position, the second position being different from the first position.

By configuring the switch arrangement as described above, the tolerance requirements on the different components of the device may be significantly lowered whereby the device manufacturing costs can be reduced.

In further embodiments according to the second aspect, the switch arrangement is configured to include hysteresis. In still further embodiments, the switch arrangement is configured as a linear sensor. In still further embodiments, the injection device mechanism is configured for providing an intermediate mode between dose administration mode and dose setting mode wherein the device mechanism is locked both with respect to drive member movements and dose selector movements.

In some embodiments the medical injection device includes a spring means configured for storing energy during a pre-injection procedure, and wherein the drive member, during dose administration, is forced by the spring means to expel medication from the container when energy previously stored in said spring means is released.

Also, in some embodiments, the medical injection device may be adapted to detect that an occluded needle is attached to the medical injection device. In such device, the dosing actuator may be movable from the first state to the second state by moving the dosing actuator in a distal direction. The switch arrangement and the controller is further being adapted to provide an electronic representation of a transition to a third position where said third position being positioned distally from said second position. The injection device is adapted to detect a blocked needle condition derived from said positional information of the drive member and from the position of the dosing actuator. Said needle blocked condition is only detected responsive to the dosing actuator being pressed distally relative to the third position.

In accordance with the THIRD aspect of the invention, a manual medical injection device is provided for setting and injecting doses of a medicament, the injection device comprising:
a housing,
a dosage selector being operable to set the amount of a dose by rotating the dosage selector relatively to the housing,
a dose display for displaying the amount of a set dose as set by the dosage selector,
an electronic storage having a plurality of storage entries, each storage entry adapted for storing a respective preset dose amount,
electronic signaling means for electronically generating a plurality of different preset dose indications, each of said preset dose indications being assignable to a respective one of said stored preset dose amounts,
   wherein the electronic signaling means are adapted to generate a respective one of said plurality of different dose indications exclusively when the dosage amount as being set by means of the dosage selector correspond to one of said stored preset dose amounts. Said preset dose indication may be additional to the dosage amount as displayed on the dose display.

In order to support patients following treatment regimens in which they always take two or three fixed size doses at the same times each day, the injection device may include one, two or more preset dose sizes which are stored in the device and wherein the injection device is adapted to emit a signal to the user indicating that the selection, as dialed by dosage selector, exactly corresponds to the desired preset dose.

The dose display for displaying the amount of a set dose may be a mechanical dose dial or an electronic display.

The electronic signalling means for electronically generating a plurality of different preset dose indications may be adapted to provide the preset dose indication as a signal which can be audible and/or tactile and/or visual.

The container to be used with the injection device according to the first, second and third aspect may be a cartridge prefilled with a medicament. The container may be releasably mounted to the injection device so that the container may be exchanged with a new one when the medicament in the previous container has been exhausted. Alternatively, the container may be fixedly attached to the injection device whereby the container and the injection device is disposed off when the medicament initially contained in the injection device has been exhausted.

As used herein when referring to the first and second aspect of the invention, the term “manual injection device” defines a non-motorized injection device, i.e. a type of device not incorporating an electric motor and where the energy for carrying out the injection procedure is delivered by the user, such as during the injection operation and/or before the injection operation. Non-limiting examples of manual injection devices include injection devices where the user actively pushes forward a dosing button throughout the injection procedure as well as injection devices where the user winds a spring-mechanism to accumulate energy during a pre-injecting procedure, such as during dose setting, and where the accumulated energy is used to press forward the drive means of the injection device during the dose administration. The term dose setting mode defines a state for the injection device, in which the dose to be adminstered may be adjusted and in which substantial movements of piston rod and hence an expelling operation is not possible. The term dose administration mode defines a state for the injection device, in which expelling of a dose is possible, and in which an adjustment of a dose is not possible.

### BRIEF DESCRIPTION OF THE DRAWINGS

In the following the invention will be further described with references to the drawings, wherein

FIG. 1a and FIG. 1b show respectively a cross sectional view and a top view of a prior art injection device,

FIG. 2a and FIG. 2b show respectively a cross sectional view and a top view of an example injection device according to the first aspect of the invention,

FIG. 3 is a graph which shows defined mechanical mode locks as a function of dosing actuator position of the injection device shown in FIGS. 2a and 2b,

FIG. 4 is a graph showing an electronic representation of mode switch patterns incorporating hysteresis during transition from dose setting mode to dose administration mode and vice versa,

FIG. 5 is a graph showing exemplary dose sensor signals and corresponding actuator positions during an example operation sequence of the injection device shown in FIGS. 2a and 2b, and

FIG. 6 is a schematic representation of electronic components in the device shown in FIGS. 2a and 2b.

### DESCRIPTION OF EXEMPLARY EMBODIMENTS

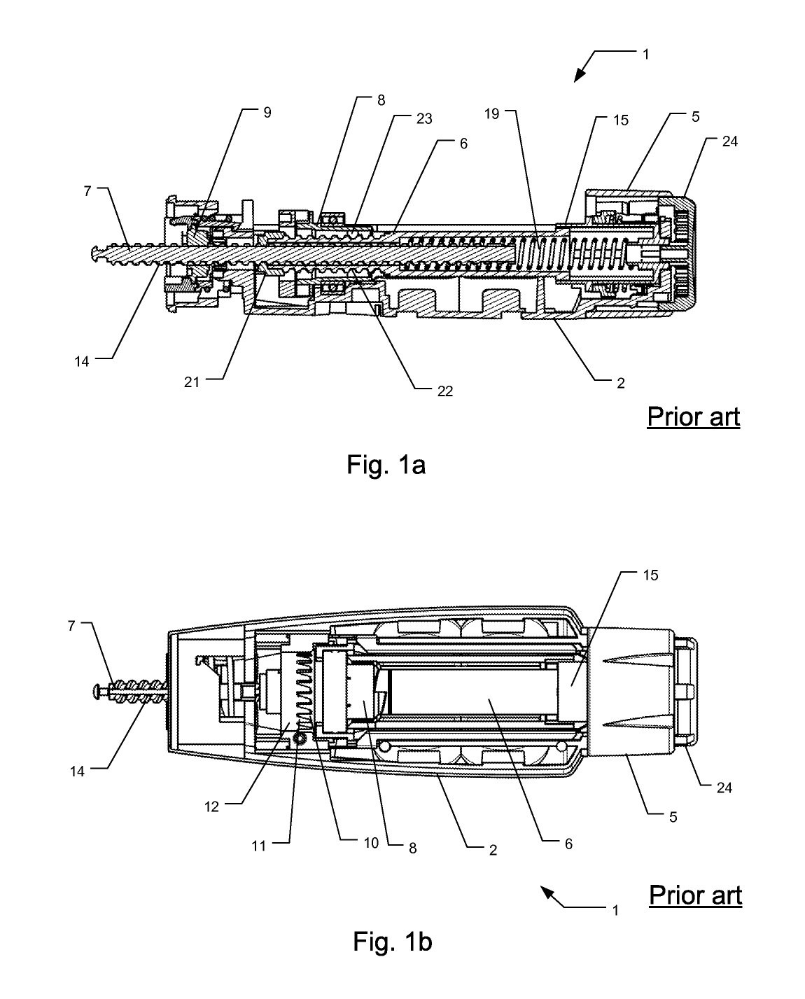

FIGS. 1a and 1b show cross-sectional and top view representations of a prior art injection device for injecting metered amounts of a liquid medication. Such device correspond to the injection device shown in FIGS. 11-15 of WO patent application No. 2008/116766, this reference being incorporated by reference in its entirety. As described in WO 2008/116766, the injection device 1 includes a housing 2 which is adapted to receive a medicament filled container. The container may be in the form of a cartridge of the type including a piercable septum in a distal end of the cartridge and a slideable rubber plunger which is initially positioned in a proximal end of the cartridge. In a situation of use, the piercable septum is to be pierced by an injection needle which connects to the cartridge end of the device. During an administering operation, a piston rod 7 of the device 1 can be forced to move towards the distal end of the device to push forward the plunger in the cartridge in order to expel a pre-adjusted dose of medication.

Actuation of the administering operation occurs by means of a dosing actuator in the form of an injection button 24, the injection button 24 being movable between a dose setting state and a dose administration state. During a dose setting operation, by manipulating a dosage selector in the form of a rotatable dose knob 5, the quantity of medication which is expelled during dose administration may initially be adjusted by the user.

In the injection device 1 of FIGS. 1a and 1b, the piston rod 7 includes an outer thread 14 which engages a cooperating inner thread 21 of a drive member formed by a dosage tube 6. During dose setting and dose administering operations, the piston rod 7 is rotationally locked with respect to the housing 2 by virtue of keyed engagement with locking item 9, the locking item 9 being rotationally fixed with respect to the housing when a cartridge is received by the housing 2.

Dosage tube 6 further comprises an outer thread 22 which engages an inner thread 23 of a locking nut 8. Locking nut 8 is mounted in the housing 2 so that locking nut 8 may rotate around a central longitudinal axis of the device while being axially fixed relative to the housing. At the distal end, locking nut 8 further comprises a set of axially extending teeth 10 adapted to engage a corresponding set of teeth 11 formed at the proximal end of a locking item 12. Locking item 12 is rotationally locked with respect to the housing and is selectively moveable between a first proximal position in which the set of teeth 11 engages the set of teeth 10 to a second distal position in which the two sets of teeth 11 and 10 do not engage. When locking item 12 is in its proximal position, due to the said engagement, locking nut 8 is prevented from rotating, whereas when locking item 12 is in its distal position, locking nut 8 can rotate freely relative to locking item 12.

The injection button 24 is connected to locking item 12 so that axial movements of the injection button is being transferred to locking item 12. The injection button 24 is biased towards a proximal position which correspond to the dose setting state and the injection button 24 is movable, upon being pressed down by a user, towards a distal position which correspond to the dose administration state in which the locking nut 8 is freely rotatable relative to locking item 12.

In the device 1, the dosage tube 6 is adapted to follow rotation of the dose knob 5 as the dose knob 5 is being rotated by a user in the selection of a proper dose size, i.e. by dialling up and down. A compressible spring 19 acts to urge dosage tube 6 in the distal direction. In FIGS. 1a and 1b the injection device is in an initial state corresponding to a state where a dose size of 0 units have been selected and where the injection button 24 is not pressed down. During subsequent increasing of the dose size, the dosage tube 6 is moved axially towards the proximal end of the device, in accordance with rotation of dose knob 5 and in accordance with the threaded connection 22 and 23. During the dose setting operation, as the dosage tube 6 is moved proximally, energy is accumulated in compressible spring 19, which subsequently, during the dose administration operation, will be released for driving forward the dosage tube 6 and consequently also piston rod 7.

As observed in WO 2008/116766, the dose injection button 24 may comprise a set of teeth which engages with cooperating teeth of the housing 2 when the injection button 24 is pushed down, i.e. when the injection button is in its distal position. As injection button 24 is adapted to follow rotational movements of dose knob 5, dose knob 5 is not allowed to rotate during dose administration. Hence, the dose size cannot be altered by manipulating the dose knob 5, when the injection button 24 is in the dose administration state.

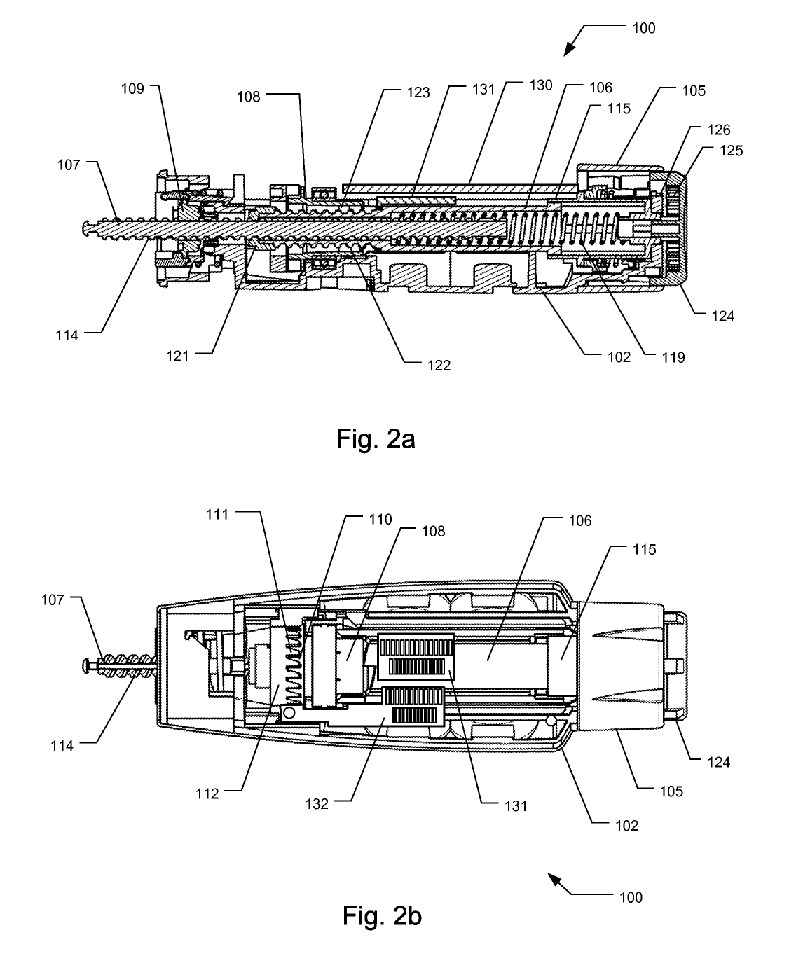

Turning now to FIGS. 2a and 2b, an injection device 100 according to the first and second aspect of the present invention is shown in a cross-sectional view and in a top view respectively. The operation of the device 100 of FIGS. 2a and 2b correspond to the operation of the device 1 shown in FIGS. 1a and 1b. Corresponding parts of the device 100 are given corresponding reference numbers with a prefixed “1”, ie. dosage tube 6 of the device 1 corresponds to dosage tube 106 of the device 100 etc. To improve clarity, some of the parts of the injection device 100 have been omitted from the figures in order to show parts arranged in the interior of the injection device.

The injection device 100 is adapted to operate in a dose setting mode where a dose can be selectively adjusted by rotating dose knob 105 and in an dose administration mode where the injection button 124 is pushed in to expel a preselected dose of medicament. The dose knob 105 may include an indexing mechanism (not shown) which during rotation of knob 105 provides incremental clicks for each particular dose increments. The clicks may be provided as an acoustically emitted signal or alternatively as a vibrational signal which is perceivable by a user holding the injection device 100. Exemplary dose increments may correspond to 1 IU or alternatively ½ IU.

The injection device 100 of FIGS. 2a and 2b is equipped with various electronic components providing additional operational features of the injection device. Reference is generally made to FIG. 6 which depicts schematically a cartridge to which a drive means, such as the drive member 106, is coupled and used to drive forward the piston of the cartridge. The movements of the drive means is detected by sensors coupled to a controller for monitoring the operation of the device. To the controller, a user interface is coupled for gaining access to the electronic features of the injection device, the user interface including audiovisual means and/or communication means for communicating with an external device.

The device 100 of FIGS. 2a and 2b is provided with an electronic circuitry incorporating a controller and sensors for monitoring operation of the device and for providing an electronic representation of the mechanical mode of operation, i.e. whether the device is in dose setting mode or in dose administration mode. The electronics circuitry includes a storage which is adapted to hold various types of data having regard to a number of administered dosages, such as the dosage quantities and the time stamps related to each administered dosage. By means of the storage from which stored data can be later recalled, a logbook function may be provided. The electronic circuitry comprises a suitable energy source such as a battery (not shown) for driving the electronics. The electronic circuitry may further include a display (not shown) on which set doses and/or injected doses may be shown and may incorporate a electronic sound transducer, such as a piezo-electric buzzer, to electronically generate auditive and/or vibrational signals. The stored data may be communicated by means of a communications interface to an external device for presentation on the external device.

Shown schematically in FIGS. 2a and 2b, a main electronics circuitry 130 is incorporated in the injection device 100. Main electronics circuitry 130 is mounted fixedly within housing 102. The main electronics circuitry 130 comprises a controller and driving circuitry for the sensor system. In the device 100, a single sensor arrangement is used for picking up positional data related to the movements of the dosage tube 106 as this component moves during the dose setting operation and during the dose administration operation. In order to discriminate between positional data which on the one hand correspond to movements during the dose setting operation and on the other hand correspond to movements during the dose administration operation, a switch arrangement is furthermore incorporated to detect the particular mode of the device, i.e. whether the device is in dose setting mode or in dose administration mode.

In the depicted embodiment, the sensor arrangement and the switch arrangement are provided as linear sensors for respectively monitoring axial displacement of the dosage tube 106 and axial displacement of the locking item 112/injection button 24.

In the embodiment shown in FIGS. 2a and 2b, both the sensor arrangement and the switch arrangement are provided as capacitively based linear sensors of the type generally described in U.S. Pat. No. 5,731,707. This type of sensor includes first and second substrates mounted to be moved relatively to each other, the first and the second substrates having printed patterns of electrodes disposed along an axis. The two substrates are arranged in parallel so that electric signals applied to the electrodes of the first substrate capacitively couple to the electrodes of the other of the substrates where it capacitively couples back to the first substrate for signal analysis. The relative or absolute position along the said axis between the first and the second substrate can be determined from the reflected signals in conjunction with the applied electrical signals. In alternative embodiments, other types of sensors may be used which may rely on different measurable signals, such as electrical galvanic signals, optical signals, inductive signals and other types of signals.

As shown in FIG. 2b, a first electrode pattern is arranged on a first sensor substrate 131, this substrate being coupled to an annular track of dosage tube 106 so that the substrate 131 follows axial displacements of dosage tube 106. Further, a second electrode pattern is arranged on a second sensor substrate 132 which is mounted on lock item 112, i.e. substrate 132 follows axial displacements of lock item 112 and hence also axial displacements of the injection button 124.

The first and the second electrode patterns arranged on substrates 131 and 132 cooperate with respective designated electrode patterns formed on the main electronic circuitry 130 so as to provide a sensor arrangement for monitoring dosage tube axial movements and to provide a switch arrangement for monitoring the axial movements of the injection button 124.

As regards the particular choice of the sensor arrangement 130/131, the capacitively based position sensor provides a fine resolution which may be in the order such as 1/10, 1/50 or 1/100 of a corresponding incremental step which can be operated by the indexing mechanism of the dose knob 105 and displayed on the injection device 100 or logged in a diary of the device.

In the depicted embodiment shown in FIGS. 2a and 2b, the injection button 124 includes a set of teeth 125 which engages corresponding set of teeth 126 formed in the housing 102. As the injection button 124 is rotationally connected to the dose knob 105 by means of axially extending splines, the rotational position of dose knob 105 is prevented from being altered when injection button is pressed down, i.e. during the dose administration procedure. As described above, when the injection button 124 is in its proximal position, the locking nut 108 is prevented from rotating due to its engagement with locking item 112. By appropriately designing the pitch of the two thread connections of the dosage tube 106 (inner thread 121 and outer thread 122) the piston rod 107 is prevented from moving when dosage tube 106 is rotated during dose setting. Hence, dose administration is prevented during the dose setting procedure.

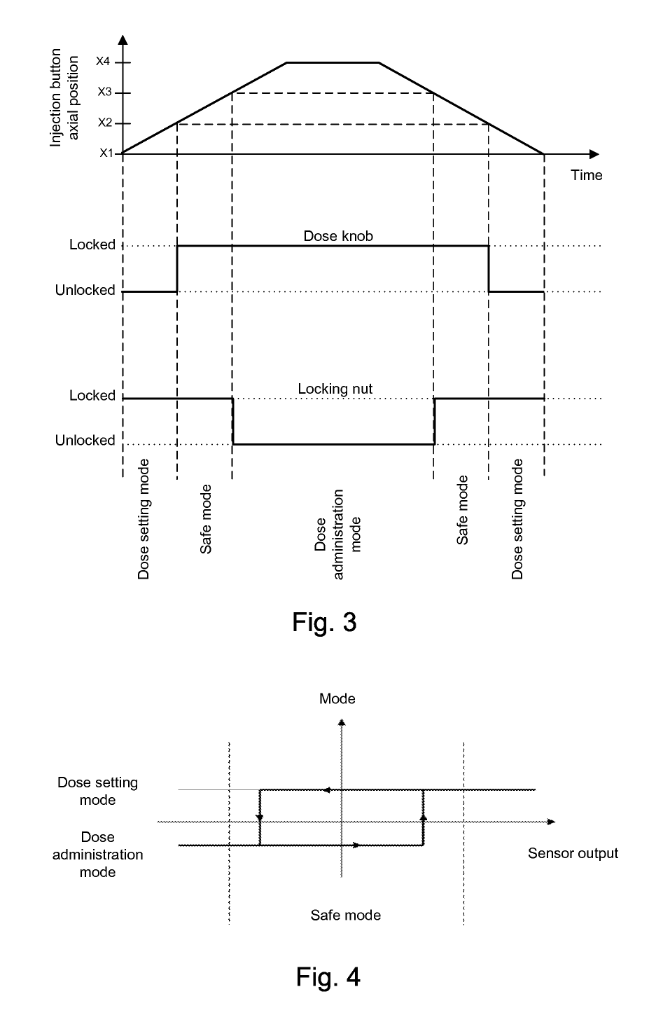

In the exemplary embodiment shown in FIGS. 2a and 2b, the axial extension of toothed connections 110/111 and 125/126 are so designed that in a range of intermediary axial positions of injection button 124 between dose setting mode and dose administration mode, a third intermediary mode is defined where both dose administration and dose setting is prevented, this intermediary mode being designated “safe mode”. Reference is made to FIG. 3, which depicts a graphical representation of the injection device modes as a function of axial position of the injection button 124. When the injection button 124 is positioned in the range of axial positions X1 to X2, the dose knob 105 is allowed to rotate in order to set a dose. When the injection button 124 is positioned in the range of axial positions X2 to X4, the dose knob 105 is prevented from rotating. Only when the injection button 124 is positioned in the range of axial positions X3 to X4, the lock nut 108 is able to rotate. Hence, when injection button 124 is positioned in the range of axial positions X1 to X3, the lock nut 108 is rotationally locked by lock item 112 and the piston rod is prevented from moving axially. As the injection button 124 is moved from its proximal position X1, in which the device is in dose setting mode, towards its most distal position X4, the injection button passes a position X2 where the mechanism of the injection device enters safe mode where it remains until passage of position X3. After passage of X3 the device enters dose administration mode corresponding to the injection button 124 being pressed in. Subsequently, as the injection button 124 is returned to its initial position X1, it passes a range of positions from X3 to X2 where the injection device is in safe mode. At these intermediary positions of the injection button 124, the dose setting and delivery mechanism is mechanically locked, thereby providing an accurate and reliable mechanism having consistent transitions between the mechanical operational modes of the device.

The output from the switch arrangement 130/132 may in some embodiments be adapted to provide a single threshold position in which the electronic representation of the operational mode of the device discriminates between dose setting mode and dose administration mode. By using a linear sensor for the switch sensor arrangement a switch arrangement can be provided wherein the electronic representation of the threshold position between dose setting mode and dose administration mode may be programmed into the controller to take into account the mechanical tolerances of a batch of production samples or even the tolerances of each particular production sample. Such programming may appropriately be carried out by programming the software designed to be executed by the controller. When the device is so adapted that the injection button 124 travel include a safe mode region, the threshold position may be positioned in that safe mode region, such as close to the mechanical transition point between safe mode and dose setting mode.

In the depicted embodiment, the switch arrangement 130/132 is adapted to provide an electronic representation of the operational mode by assigning a first threshold position for the detection of the mode change from dose setting mode to dose administration mode and assigning a second threshold position for the detection of the mode change from dose administration mode to dose setting mode. As depicted in FIG. 4, hysteresis may be incorporated into the switch arrangement so as to provide an increased accuracy in the detection of mode changes. In such embodiment the transition point assigned to the mode change from dose setting mode to dose administration mode is selected in the safe mode region near position X3 (see FIG. 3) whereas the transition point assigned to the mode change from dose administration mode to dose setting mode is selected in the safe mode region near position X2. In this manner, mode changes can be detected with improved reliability ensuring synchronization between particular mechanical mode changes and the electronic representation thereof. In particular, in applications where power consumption is to be minimized, the sampling frequency for sampling the signals derived from the switch arrangement may be chosen comparatively low. In such systems, the switch arrangement 130/132 may be implemented with hysteresis to thereby provide a system having a high degree of precision.

The sensor arrangement 130/131 provides positional information regarding the axial position of the dosage tube 106 during operation of the device. As the dosage tube 106 moves in the proximal direction during dose setting when dialling up, and moves in the distal direction when dialling down, the extent of movement detected by the sensor arrangement 130/131 is associated with the a particular dose size as adjusted with the dose knob 5.

In the embodiment of FIGS. 2a and 2b, the dosage tube 106 moves in the distal direction during dose administration. Hence, the sensor arrangement 130/131 may be designed to provide positional information regarding the axial position of the dosage tube 106 during the dose administration procedure, thereby offering the monitoring and read-out of the instant expelled dose quantity even during the dose administration movement. Hence, the device may be halted at any time during dose administration and the expelled quantity may be shown on the display (not shown) of the device.

In the device 100, since movements of a single component (dosage tube 106), which both moves axially during dose setting and during dose administration, is only being monitored in one and the same dimension, the switch arrangement 130/132 is required for properly discriminating whether a particular distal movement of the dosage tube 106 is caused by an actual expelling movement or alternatively is caused by the user dialling down an initially set dose.

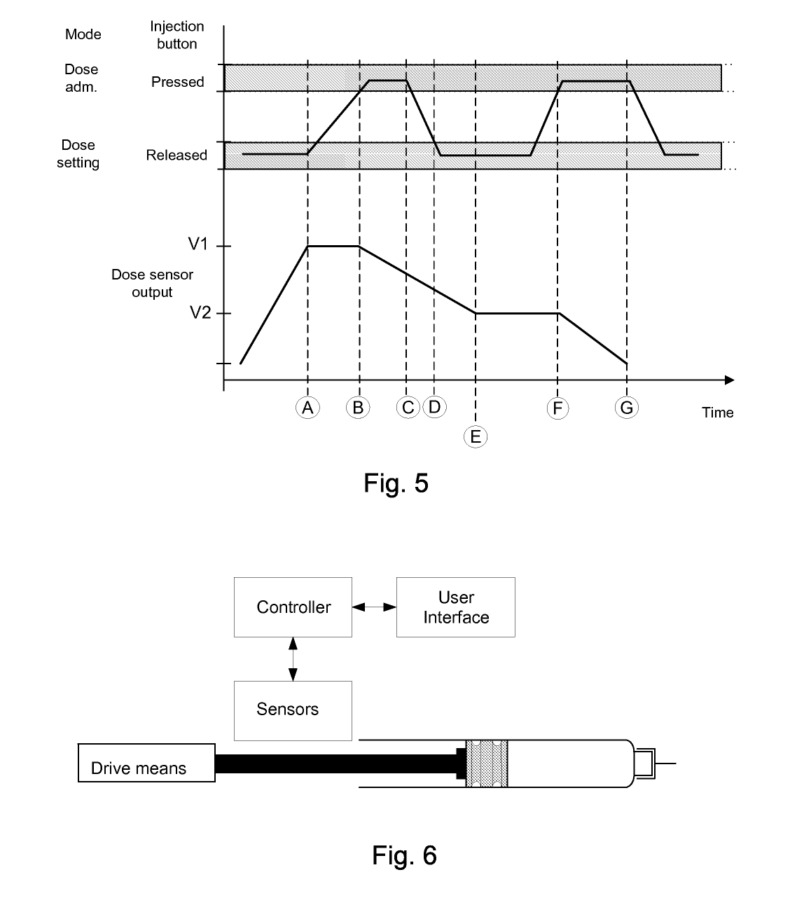

FIG. 5 depicts a schematic representation of an example operating sequence over time of the device shown in FIGS. 2a and 2b. In that sequence a dose is initially set, then, during dose administration, the dose button is released so that a partial expelling of the set dose is carried out, i.e. the expelling is interrupted for a given time, and lastly the dose button is pressed in again to continue the dose administration for completion of the expelling of the dose amount as set.

The electronic representation of the operational mode during the example operational sequence is shown in the upper part of FIG. 5. The upper grey area corresponds to dose button positions wherein the mechanical mode of the injection device is dose administration. The lower grey area corresponds to dose button positions wherein the mechanical mode of the injection device is dose setting. The intermediate area between the two grey areas correspond to safe mode. The curve in the upper part of FIG. 5 represents signals picked up by the switch arrangement 130/132 during the said operating sequence. The lower part of FIG. 5 depicts sensor values recorded by the dose sensor arrangement 130/131 which corresponds to the position of the dosage tube 106 throughout the example operational sequence.

At time “A” a dose has been set by dialling up the dose knob 105 to a particular dose size. The corresponding dose sensor output is V1. At time “A” the injection button 124 is pressed down which causes the injection device mechanism to enter into dose administration mode at time “B”. Due to the pressure exerted by spring 119 the dosage tube 106 is forced forward in the injection device. At time “C” the injection button 124 is released for the purpose of pausing the injection. This causes the device mechanism to enter into dose setting mode at time “D”. In this state no rotation of the dose knob is carried out. However, as apparent in the lower part of the figure, the dose sensor output continues to change until time “E”, where the actual forward movement of dosage tube 106 has ended corresponding to a dose sensor output V2. After a pause, the injection button 124 is pressed in again causing the device to enter dose administration mode at time “F”. The injection button 124 is maintained in the dose administration state until end-of-dose where the injection button 124 is released at time “G” causing the injection device to enter into dose setting mode again.

As mentioned above, the dosage tube 106 moves from time “C” to time “E”, even though the teeth 111 of lock item 112 partly or fully engages the teeth 110 of locking nut 108. From time “D” to time “E” the electronic signals picked up by switch arrangement 130/132 indicates that the operational mode of the pen is dose setting mode, but the dosage tube 106 moves slightly even though the dose knob 105 has not been rotated during this interval. The slight movement of dosage tube 106 during interval “C” to “E” is caused by dynamic effects during transition from one operational mode to the other. In the particular device shown in FIGS. 2a and 2b, the spring 119 causes a pressure build-up which continues to force forward dosage tube 106 until slack between components, elastic elongation/compression of components etc. have been stabilized, i.e. when the mechanism has been fully locked by locking nut 108.

The shift from dose administration mode to dose setting mode may be designed in the electronics to occur very close to the mechanical transition from safe mode to dose setting mode.

However, by introducing a time lag counted from the time of transition into dose setting mode, changes in dose sensor values occurring shortly after the transition into dose setting mode may be taken into consideration when determining the expelled dose. In the depicted embodiment the time delay is chosen to be 400 milliseconds. Other embodiments may incorporate time delays of other magnitudes. The time delay may be provided by appropriately programming the delay in software, i.e. so that the time delay is determined by an electronic counter.

Other embodiments may include a switch arrangement wherein the switch arrangement is designed to provide a switch signal at a single transition point to discriminate between dose administration mode and dose setting mode. In such system, the transition point may be assigned to be close to the mechanical transition point where the mechanical mode changes from safe mode to dose setting mode.

In one embodiment, the device 100 may be configured to detect a needle blocked condition, by means of the method as disclosed in WO 2009/083600. In such a system, during injection, if the speed as sensed by monitoring the positional information relating to the dosage tube 106, i.e. by means of the dose sensor arrangement 130/131, is below a first threshold speed, this condition may be deemed to involve an occlusion downstream from the reservoir. In particular, if no movement of the drive member is detected during injection, the speed data as derived from the sensor contains a speed corresponding to no movement of the drive member and this triggers a control action such as an alarm indicating that the needle may be occluded. In case that a dose has not been selected (i.e. that the dosage knob 104 is set to 0 units) and that the dosing actuator is pressed in, in order not to emit a false blocked needle signal, the positional information relating to the dosage tube 106 indicating that no dose has been selected may be used to filter out such false alarms. Further, while referring to FIG. 3, in order to incorporate the above described blocked needle functionality, the switch arrangement 130,132 may be configured to provide a further threshold position X3′ (not shown) between the position X3 and X4, where X3′ is positioned close to X3 but situated sufficiently far from X3 to ensure that X3′ is positioned in a region where the locking nut 108 is disengaged from the locking item 112.

In some embodiments, the injection device 100 may include an electrical sound transducer such as a piezo-electric buzzer or other means for electrically generating acoustic signals and/or vibrational signals. The piezo-electric buzzer may be used for electronically generating dose administration signals by generating repetitive sound signals or alternatively a continuous sounding signal being emitted as long as the dose sensor arrangement 130/131 detects that the dosage tube 106 moves during dose administration. In this way, the user of the device is provided with adequate response from the injection device 100, signalling that a dose is actually being expelled. The repetitive sound signals may be emitted as a series of click sounds or beeps where the repetition frequency of each click or beep may substantially correspond to the expelling of each unit or half-unit dose, for example in accordance with the chosen incremental step for the rotational movements of dose knob 105. The piezo-electric buzzer may also be used for generating other signals or alarms, such as signalling the incremental steps during operation of dose knob 105, the appropriate time for removing the needle equipped device from the skin of the user subsequent to an injection administration, the sensing of a blocked needle condition as well as other alarm or error conditions. The above described electronically generated dose administration signal may also be used in other devices, such as in other manual injection devices as defined in the context of the present application. As opposed to conventional manual injection devices including an injection clicker sounding during the expelling procedure (where the conventional mechanically generated clicks require mechanical energy), the provision of the above described electronically generated dose administration signal are particularly beneficial as this may reduce the injection force required for expelling the medicament or alternatively the force required for straining a spring in a spring-operated injection device.

In order to support patients following treatment regimens in which they always take two or three fixed size doses at the same times each day, the injection device 100 may include one, two or more preset dose sizes which are stored in the device and wherein the injection device 100 is adapted to emit a signal to the user indicating that the selection, as dialled by dose knob 105, exactly corresponds to the desired preset dose. The signal can be audible, tactile and/or visual. In the shown embodiment, the injection device is adapted to store three individual preset dose sizes each of which are associated their own distinctive signal which is different from any other signal emitted by the device. For example, the first preset dose may be associated with a rising sun symbol (indicating a morning administration) which appear on the display when the particular dose size corresponding to the value stored in the first preset dose is selected during operation of the dose knob 105. The displaying of the first preset dose may be accompanied by a single short beep emitted by the buzzer. Similarly, the second preset dose may be associated with a sun symbol shown on the display (indicating a noon administration) which may be accompanied by two short beeps. The third preset dose may accordingly be associated with the displaying of a moon symbol (indicating an evening administration) which may be accompanied by three short beeps.

The programming and storage of the above described preset doses may be performed during production, during a visit at the general practitioner or by the user himself. The programming may for example be performed by using the dose knob 105 and/or the injection button 124. The set-up of the preset doses may be initialized by holding the injection button in its pressed-in state for a prolonged time while the dose knob 105 is adjusted to its 0 unit position. Subsequently, the first preset dose may be selected by use of the dose knob 105 and the selection will be stored by pressing in injection button 124, then the second preset dose and the third preset dose may be selected and stored in succession in a similar way. As an alternative to the above procedure, the initializing of the set-up may be provided by dialling the dose knob 105 in a particular sequence of dose sizes in succession, such as dialling 0-10-0-20-0-30-0 or the like. Still alternatively, the programming and storage of the preset doses may be performed using an external device equipped with suitable input means and in communication with the injection device 100.

The above described preset dose functionality may also be used in other types of manual injection devices, such as manual injection pens having a dosing button which the user presses manually down for expelling the selected dose and having a rotatable dosage selector, and where a preset dose is signalled upon dialling in a dosage amount which correspond to the amount stored for that particular preset dose.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents. This invention includes all modifications and equivalents of the subject matter recited in the claims appended hereto as permitted by applicable law.

1. A medical injection device for injection of set doses of a medicament from a medicament container, the injection device being adapted for operating in a dose setting mode and in a dose administration mode, the injection device comprising:
a drive member which moves during dose setting mode and/or during dose administration mode,
a dose sensor arrangement adapted to sense positional information associated with the drive member,
a dosing actuator reversibly actuatable from a first state for operating the device in the dose setting mode to a second state for operating the device in the dose administration mode,
a switch arrangement adapted to sense positional information associated with the dosing actuator, and
a controller coupled to the dose sensor arrangement and to the switch arrangement, the controller being adapted to provide an electronic mode representation of the mode of operation and being adapted to determine the size of a set dose and/or an expelled dose,
wherein responsive to the dosing actuator moving from the second state to the first state, the electronic mode representation is configured to enter dose setting mode after a time delay following detecting the dosing actuator has moved away from said second state or detecting the dosing actuator has moved into said first state and wherein the size determination of a set dose and/or an expelled dose is based on the electronic mode representation.

2. The medical injection device as defined in claim 1, wherein the dose sensor arrangement comprises a single sensor adapted to detect said positional information associated with the drive member during both dose administration and dose setting operations.

3. The medical injection device as defined in claim 1, wherein the controller is adapted to determine the size of a set dose and adapted to determine the size of an expelled dose.

4. The medical injection device as defined in claim 1, wherein the device further comprises a spring means configured for storing energy during a pre-injection procedure, and wherein the drive member, during dose administration, is forced by the spring means to expel medication from the container when energy previously stored in said spring means is released.

5. The medical injection device as defined in claim 4, wherein the dosing actuator cooperates with the spring means to release energy when the dosing actuator is moved into its second state, said release of energy being interrupted when the dosing actuator is returned towards its first state.

6. The medical injection device as defined in claim 1, wherein the time delay is larger than 5 msecs.

7. The medical injection device as defined in claim 1, wherein the injection device includes a dosage selector which is adapted to move in incremental steps and wherein the resolution of the sensor arrangement is higher than a corresponding incremental step.

8. A medical injection device for injection of set doses of a medicament from a medicament container, the injection device being adapted for operating in a dose setting mode and in a dose administration mode, the injection device comprising:
a drive member being adapted to expel a dose of medicament from the medicament container during dose administration,
a dose sensor arrangement adapted to detect positional information of the drive member,
a dosing actuator reversibly movable from a first state for operating the device in the dose setting mode to a second state for operating the device in the dose administration mode,
a switch arrangement monitoring the position of the dosing actuator, and
a controller coupled to the dose sensor arrangement and to the switch arrangement to determine the size of a set dose,
wherein the switch arrangement and the controller is adapted to provide an electronic representation of transition into dose setting mode at a first position and to provide an electronic representation of transition into dose administration mode at a second position, the second position being different from the first position.

9. The medical injection device as defined in claim 8, wherein the device further comprises a spring means configured for storing energy during a pre-injection procedure, and wherein the drive member, during dose administration, is forced by the spring means to expel medication from the container when energy previously stored in said spring means is released.

10. The medical injection device as defined in claim 9, wherein the dosing actuator is movable from the first state to the second state by moving the dosing actuator in a distal direction, the switch arrangement and the controller further being adapted to provide an electronic representation of a transition to a third position, said third position being positioned distally from said second position and wherein the injection device is adapted to detect a blocked needle condition derived from said positional information of the drive member and from the position of the dosing actuator, said needle blocked condition being only responsive to the dosing actuator being pressed distally relative to the third position.

11. The medical injection device as defined in claim 8, wherein the switch arrangement comprises a linear sensor.

12. (canceled)
